(19) United States
(12) Patent Application Publication
(10) Pub. No.: US 2022/0176122 A1
(43) Pub. Date: Jun. 9, 2022

(54) SUPPORT STRUCTURE FOR A WEARABLE MEDICAL DEVICE WITH ADJUSTABLE FASTENER

(71) Applicant: West Affum Holdings Corp., Grand Cayman (KY)

(72) Inventors: Laura M. Gustavson, Redmond, WA (US); Joseph L. Sullivan, Kirkland, WA (US)

(21) Appl. No.: 17/246,465

(22) Filed: Apr. 30, 2021

Related U.S. Application Data
(60) Provisional application No. 63/122,009, filed on Dec. 7, 2020.

(51) Int. Cl. A61N 1/362; A61N 1/365; A61N 1/39

(52) U.S. Cl. CPC A61N 1/3625; A61N 1/36592; A61N 1/3968; A61N 1/3621; A61N 1/3904; A61N 1/36542

(57) ABSTRACT

A wearable cardioverter defibrillator (WCD) system includes a support structure having an adjustable fastening assembly to fasten portions of the support structure together to fit the support structure on a patient. The adjustable fastening assembly enables the patient wearing the support structure to adjust the fit of the support structure without unfastening the portions from each other. The adjustable fastening assembly may also enable the patient to adjust the fit of the support structure while wearing clothing over the support structure and without having to adjust the clothing to view or access the adjustable fastening assembly.

### CROSS REFERENCE TO RELATED PATENT APPLICATIONS

This patent application claims benefit of U.S. Provisional Patent Application No. 63/122,009 filed on Dec. 7, 2020 entitled “SUPPORT STRUCTURE FOR A WEARABLE MEDICAL DEVICE WITH ADJUSTABLE FASTENER”, the disclosure of which is hereby incorporated by reference for all purposes.

### BACKGROUND

Wearable medical devices may include one or more sensors or other components that are placed onto or close to the wearer's body. For example, a person suspected of having an arrhythmia risk may be provided with a wearable medical device called a wearable cardiovert defibrillator (WCD). For example, when a person suffers from some types of heart arrhythmias, the result may be that blood flow to various parts of the body is reduced. Some arrhythmias may even result in a Sudden Cardiac Arrest (SCA). SCA can lead to death very quickly, e.g., within 10 minutes, unless treated in the interim. A doctor may recommend that this person receive an Implantable Cardioverter Defibrillator (“ICD”). The ICD is surgically implanted in the chest, and continuously monitors the person's electrocardiogram (“ECG”). If certain types of heart arrhythmias are detected, then the ICD delivers an electric shock through the heart.

After being identified as having an increased risk of an SCA, and before receiving an ICD, this person may be given a WCD system. Currently available WCD systems typically includes a support structure (e.g., as a harness, vest, or other garment) having hooks or clips for fastening the support structure when worn by the patient. The system includes a defibrillator and external electrodes, which are attached on the inside of the harness, vest, or other garment. When a patient properly wears a WCD system, the external electrodes may then make good electrical contact with the patient's skin, and therefore can help monitor the patient's ECG. If a shockable heart arrhythmia is detected, then the defibrillator of the WCD system delivers the appropriate electric shock through the patient's body, and thus through the heart.

### BRIEF SUMMARY

In accordance with some aspects of this disclosure, a wearable medical device (non-limiting examples include WCDs, Holter monitors, cardiac event monitors, etc.) is configured with a support structure having an adjustable fastening assembly. For example, in some embodiments, the adjustable fastening assembly is configured to enable easier and/or more granular adjustment of the support structure while the patient is wearing the medical device under his or her clothes. In some embodiments, the adjustable fastening assembly includes a ratchet mechanism. In some other embodiments, the adjustable fastening assembly includes a cinch mechanism. In some embodiments, the adjustable fastening assembly includes a resilient latching mechanism.

In accordance with some other aspects of this disclosure, a support structure (e.g., a garment, vest, harness, or other type of clothing for use with a wearable medical device) includes an adjustable fastening assembly to adjust the fit of the support structure while being worn by a patient. For example, in some embodiments, the adjustable fastening assembly is configured to enable easier and/or more granular adjustment of the support structure while the patient is wearing the medical device under his or her clothes. In some embodiments, the adjustable fastening assembly includes a ratchet mechanism. In some other embodiments, the adjustable fastening assembly includes a cinch mechanism. In some embodiments, the adjustable fastening assembly includes a resilient latching mechanism.

The foregoing brief summary is illustrative only and is not intended to be in any way limiting. In addition to the illustrative aspects, embodiments, and features described above, which need not all be present in all embodiments of the inventions disclosed herein, further aspects, embodiments, and features are set forth in the drawings and the following detailed description.

### BRIEF DESCRIPTION OF THE DRAWINGS

FIG. 1 is a diagram of a support structure of a WCD.

FIG. 2 is a diagram of a support structure of a WCD, with an adjustable fastening assembly schematically illustrated, according to embodiments.

FIG. 3 is a diagram an adjustable fastening assembly of the support structure of FIG. 2, according to embodiments.

FIG. 4 is a diagram an adjustable fastening assembly of the support structure of FIG. 2, according to other embodiments.

FIGS. 5A-5E are diagrams of an adjustable fastening assembly of the support structure of FIG. 2, according to still other embodiments.

FIG. 6 is a schematic diagram of components of an example wearable cardioverter defibrillator (WCD) system, according to embodiments.

FIG. 7 is a block diagram of sample components of an external defibrillator, such as the external defibrillator depicted in FIG. 6, according to embodiments.

### DETAILED DESCRIPTION

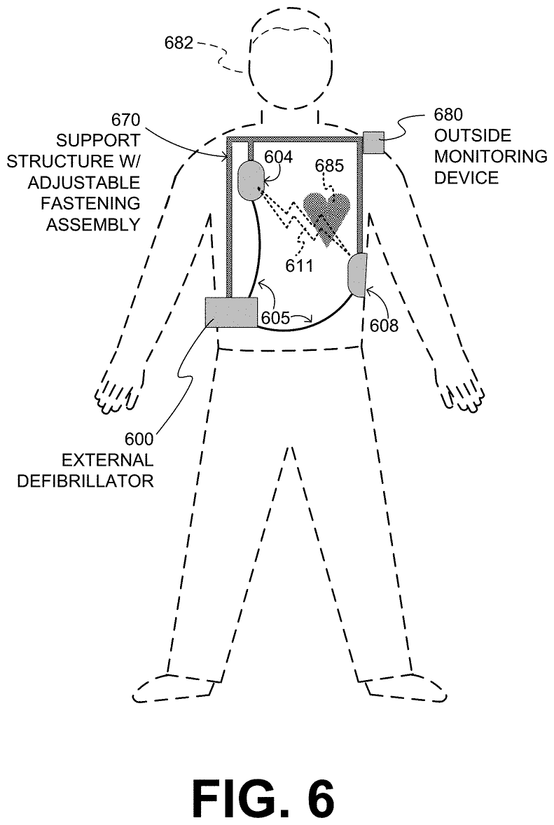

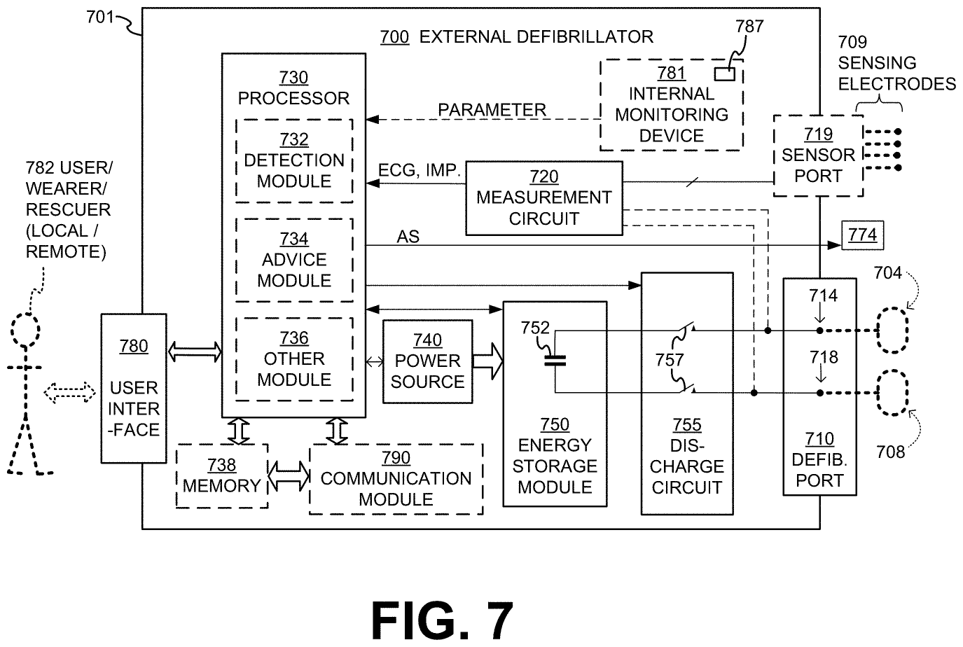

Disclosed are embodiments of wearable medical devices and systems with support structures having adjustable fastening assemblies. In embodiments the adjustable fastening assemblies are configured to enable operation by the patient while wearing the support structure under clothing. In embodiments the adjustable fastening assemblies are configured to enable operation by patients that have difficulty with the hooks and clips used for fastening support structures in currently available WCDs. Wearable medical devices include WCDs, as well as cardiac monitors such as Holter monitors, cardiac assist devices, wearable cardiac event monitors, etc. WCD embodiments are described below in conjunction with FIGS. 6 and 7.

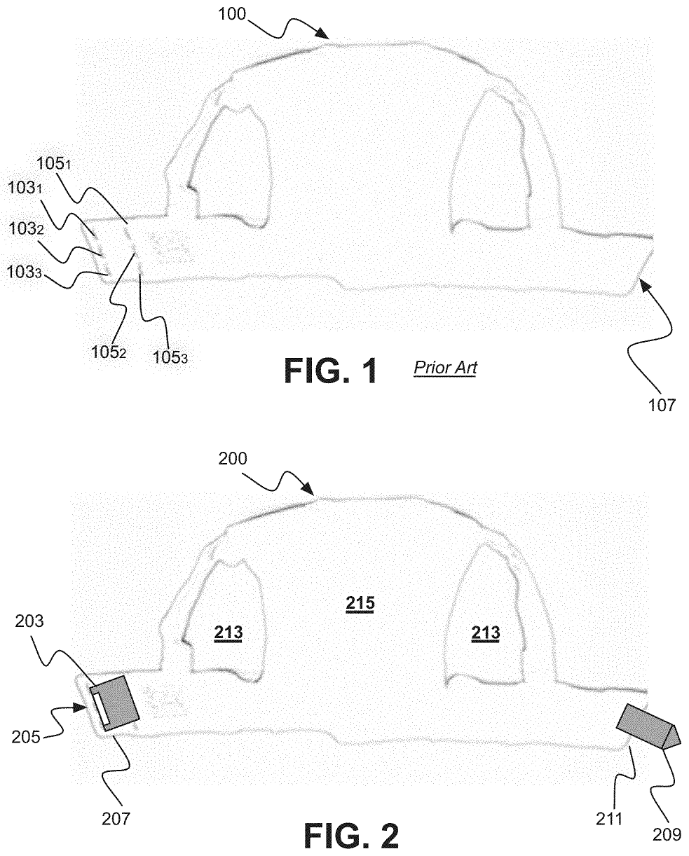

FIG. 1 is a diagram of a support structure 100 of an example existing WCD. Garments for some existing WCDs have a limited number of snap or clasp fasteners to adjust the fit of the garment on the patient. Support structure 100 includes a first column of three clips 103₁-103₃ and a second column of three clips 105₁-105₃. At a tab portion 107 of support structure 100, there are three corresponding hook-like structures (not visible in this view) that fit into a column of clips (either clips 103₁-103₃ or clips 105₁-105₃. Accordingly, there are only two possible “sizes” for support structure 100. In addition, a patient with hand strength or dexterity issues may have difficulty attaching and/or detaching the clips and hooks. Still further, changing the “size” of support structure 100 by detaching from one column of clips to a different column of clips would be difficult after the patient puts on clothing over support structure 100.

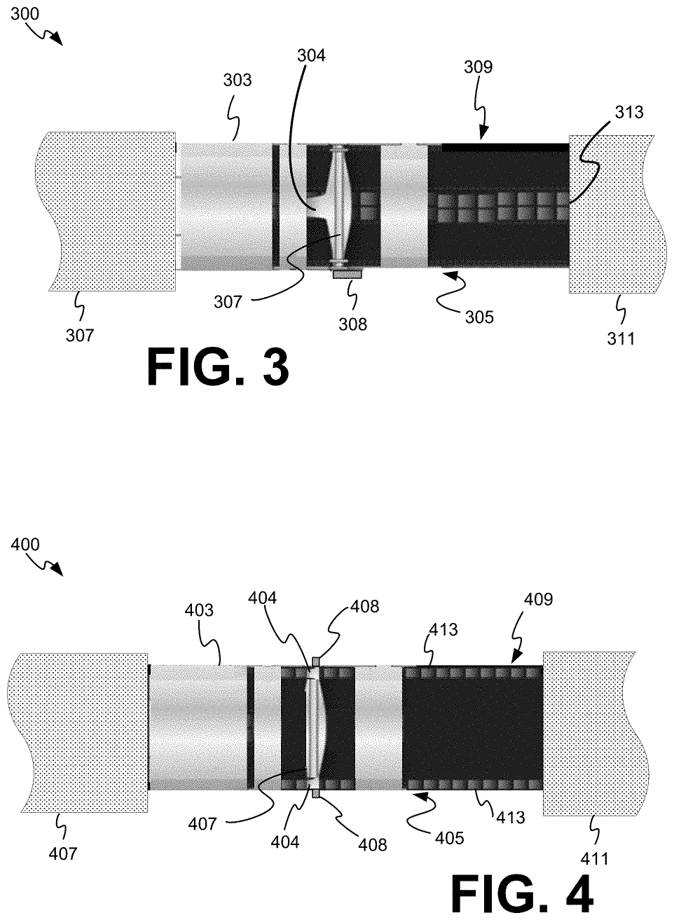

FIG. 2 is a diagram of a support structure 200 for a WCD (examples are shown in FIGS. 6 and 7), with an adjustable fastening assembly schematically illustrated, according to embodiments. Instead of the two columns of clips of support structure 100 (FIG. 1), support structure 200 includes an adjustable fastening assembly that includes a receptacle 203 with an opening 205 attached at a portion 207 of support structure 200, and a member 209 attached at a portion 211 of support structure 200. A patient can wear support structure 200 so that the WCD's sensor and therapy components are appropriately disposed on the patient. To wear support structure 200, the patient would place his or her arms through openings 213 so that a portion 215 covers the patient's back, and then fasten portions 207 and 211 together at the front of the patient's waist or torso using receptacle 203 and member 209 of the adjustable fastening assembly. Although a single receptacle and single member are shown in FIG. 2, in other embodiments one or more additional receptacles and corresponding members may be attached to portions 207 and 211, respectively. In still other embodiments, receptacle 203 can include a retainer (not shown) to help keep member 209 in place (for example if member 209 extends through the opposite side of receptacle 203 when adjusted by the patient. Embodiments of an adjustable fastening assembly are described below in conjunction with FIGS. 3-5.

In accordance with aspects of the present disclosure, in some embodiments member 209 is configured to be plugged or inserted into opening 205 of receptacle 203. In some embodiments, opening 205 extends completely through receptacle 203 so that a “forward” portion of member 209 can extend out through the other side of receptacle when member 209 is inserted into receptacle 203.

Receptacle 203 is configured to releasably retain member 209 so that receptacle 203 is configured to releasably retain member 209 so that the portions 207 and 211 are fastened together. Further, a position or a length of member 209 when retained in receptacle 203 can be adjusted by the patient while wearing support structure 200 so that the fit of support structure 200 can be adjusted. In embodiments, a position, or a length of member 209 in receptacle 203 can be adjusted to a finer granularity than the two positions of support structure 100 (FIG. 1). Further, unlike support structure 100 (FIG. 1) in which the clips and hooks need to be disengaged to change to a different column of clips, the fit of support structure 200 can be adjusted without removing member 209 from receptacle 203. This feature can be advantageous for patients trying to adjust the fit of support structure 200 when wearing it under clothing. In contrast, if a patient wearing clothing over support structure 100 (FIG. 1) tries to adjust the fit of support structure 100 and inadvertently releases the clips and/or hooks portions, the patient may have difficulty regripping those portions without moving his or her clothing out of the way (e.g., unbuttoning or lifting up a shirt or blouse), which can be undesirable in public settings.

FIG. 3 is a diagram an adjustable fastening assembly 300, which can be used with support structure 200 (FIG. 2), according to embodiments. In these embodiments, adjustable fastening assembly 300 is a ratchet-type assembly that includes a receptacle 303 and a member 309. In some embodiments, receptacle 303 is similar to a belt buckle used in ratchet-type belts and is fixedly attached to a portion 307 of the support structure (corresponding to portion 207 in FIG. 2). Embodiments of receptacle 303 can be from, without limitation, metal (including aluminum, brass, etc.), plastic (including polyoxymethylene or POM, nylon, polypropylene, acrylonitrile butadiene styrene or ABS, polyvinyl or PVC, thermoplastic polyester elastomer, thermoplastic rubber or TPR, fiber reinforced resin plastic product or FRP such as Duraflex®, etc.), plastic-metal hybrid materials, etc.

Member 309 is fixedly attached to a portion 311 of the support structure (similar to portion 211 in FIG. 2). In some embodiments, member 309 can be a shaped like a strap or tab with a longitudinal length of about 3 inches but can range from 1-6 inches in other embodiments. Embodiments of member 309 can be made from, without limitation, leather, or a fabric or webbing made of materials such as, for example, cotton, nylon, polyester, polypropylene, poly-praraphenylene terephthalamide such as Kevlar®, composite materials such as polyester or polyvinyl fluoride combined with ultra-high-molecular-weight polyethylene (e.g., Dyneema®), etc. Member 309 can also include a track 313 with wedge shaped teeth for implementing a ratchet action. In embodiments, track 313 can be made from metal, plastic, or hybrid plastic-metal materials such as described above for receptacle 303.

Receptacle 303 includes a pawl structure 304 attached to an axle structure 307 that is mounted between sidewalls in receptacle 303 so that axle structure 307 can rotate. Member 309 can be inserted or coupled to receptacle 303 via opening 305 (corresponding to opening 205 in FIG. 2). A lever 308 is used to rotate axle structure 307. When rotated, axle structure 307 causes pawl structure 304 to engage or disengage teeth in track 313 of member 309, depending on the direction of rotation. In some embodiments, axle structure 307 is spring loaded so that pawl structure 304 is biased to engage teeth in track 313. In other embodiments, other mechanisms can be used to engage and disengage pawl 304 from the teeth of track 313, and adjustable fastening assembly 300 may include one or more additional pawls and tracks.

In some embodiments, opening 305 extends all the way through receptacle 303. In such embodiments, member 309 can extend beyond receptacle 303 when the patient adjusts the support structure for a tight fit. For embodiments in which member 309 is made of a flexible material that may move around, droop and/or fold, receptacle 309 and/or portion 307 may include a retainer such as a loop or band to hold in place the part of member 309 that extends beyond receptacle 309.

In operation, pawl 304 is biased by a spring type mechanism (not shown) of axle structure 307 to engage a tooth to “lock” member 309 at a particular position when member 309 is positioned within receptacle 303. The patient can easily push or slide member 309 towards portion 307 to tighten the fit. To loosen the fit, the patient can press lever 308 to rotate axle structure 307 to disengage pawl 304 from the particular tooth of track 313 and push or slide member 309 back toward portion 311. The patient can then release the lever 308 when member 309 is at the desired position so that the spring mechanism causes pawl 304 to engage with another tooth of track 313 to “lock” member 309 at the desired position. The patient can easily perform such adjustments without having to look at receptacle 303 or member 309, which can be advantageous when the patient is wearing the support structure under his or her clothing. For some patients with hand strength or dexterity issues, operating the lever 308 can be easier compared to detaching and reattaching clips as required to adjust the fit of support structure 100 (FIG. 1).

The size/spacing of the teeth in track 313 determine the granularity of the length adjustments, and the length of the track determines the overall length adjustment range. In some embodiments, the size/spacing of the teeth is about ¼ inch, but in other embodiments the size/spacing can range from ⅛ inch to ½ inch. Compared to the 2 columns of clips used in other approaches, the ratchet-based fastener can provide much greater granularity and range in the length adjustment. This in turn can improve comfort and fit of the garment to increase compliance and effectiveness of the system. In addition, the increased range can help reduce costs by reducing the number of sizes/SKUs needed to service all of the different sizes of patients.

Moreover, adjustable fastening assembly 300 can advantageously enable improved operation of a wearable medical device in which sensors are positioned on the support structure to contact the patient's skin. For example, as will be describe in more detail further below, ECG electrodes 709 illustrated in FIG. 7 of an example WCD support structure are disposed on support structure. If there is movement at the sensor/skin interface (e.g., due to loosing of the support structure, patient movement/exercise, and/or movement/“rolling”/undulating of the patient's skin/subcutaneous fat), noise artifact may be introduced in the sensor output. In response to a detection of noise, the patient can more easily and discretely adjust the fit of support structure 200 (FIG. 2) using receptacle 303 and member 309 to improve sensor contact with the patient's skin, which can help to reduce noise artifact. For example, the WCD can be configured to detect ECG noise artifact and issue a prompt to the patient to adjust support structure 200. The patient may in response tighten support structure 200 using the above-described adjustable fastening assembly 300.

In some embodiments, the track is made of a durable yet flexible plastic material so that it can conform to the patient's body when the garment is tightened. Further, in some embodiments the material and/or configuration of track can be selected to provide some stiffness to the portion of the garment it is attached. This stiffness may help prevent the garment from twisting and/or folding over due to patient activity, patient position, etc. This stiffness can be advantageous because such twisting/folding may cause one or more of the ECG electrodes and/or therapy electrodes to lose contact with the patient's skin.

FIG. 4 is a diagram an adjustable fastening assembly 400 that can be used with support structure 200 (FIG. 2), according to other embodiments. In these embodiments, adjustable fastening assembly 400 is a ratchet-type assembly similar to adjustable fastening assembly 300 (FIG. 3) except that adjustable fastening assembly 400 is a “side squeeze” configuration using buttons instead of the lever 305 (FIG. 3).

In embodiments, adjustable fastening assembly 400 includes a receptacle 403 and a member 409. In some embodiments, receptacle 403 is similar to a belt buckle used in ratchet-type belts and is fixedly attached to a portion 407 of the support structure (corresponding to portion 207 in FIG. 2). Embodiments of receptacle 403 can be made from materials described above for receptacle 303 (FIG. 3).

Member 409 is fixedly attached to a portion 411 of the support structure (similar to portion 211 in FIG. 2). In some embodiments, member 409 can be implemented as described above for member 309 (FIG. 3). In some embodiments, member 409 can also include two tracks 413 disposed along opposite sides of member 409 as shown in FIG. 4. Tracks 413 can be implemented as described above for track 313 (FIG. 3).

In embodiments, as shown in FIG. 4 receptacle 403 can include two pawl structures 404 attached to an axle structure 407 that is mounted between sidewalls of receptacle 403 so that axle structure 407 can rotate. Member 409 can be inserted or coupled to receptacle 403 via opening 405 (corresponding to opening 205 in FIG. 2). One or more buttons 408 can be used to rotate axle structure 307. For example, buttons 408 may be spring loaded and engage spiral grooves in axle structure 407. When pressed, the buttons cause axle structure 407 to rotate which is turn causes pawls 404 to disengage teeth in tracks 413. When buttons 408 are released, axle structure 407 rotates back to its original position causing pawls 404 to engage with teeth in tracks 413. In some embodiments, axle structure 407 is spring loaded so that pawl structure 404 is biased to engage teeth in track 413. In other embodiments, axle structure 407 is formed of resilient and flexible material that bends when buttons 408 are pressed. The bending causes pawls 404 to disengage from the teeth of tracks 413. When buttons 408 are released, axle structure 407 resiliently returns to its original position causing pawls 404 to again engage with teeth in tracks 413. In other embodiments, other mechanisms can be used to engage and disengage pawls 404 from the teeth of tracks 413.

In some embodiments, opening 405 extends all the way through receptacle 403. In such embodiments, a portion of member 409 can extend beyond receptacle 403 when the patient adjusts the support structure for a tight fit. For embodiments in which member 409 is made of a flexible material that may move around, droop and/or fold, receptacle 409 and/or portion 407 may include a retainer such as a loop or band to hold in place the part of member 409 that extends beyond receptacle 409.

In some embodiments, the adjustable fastening assemblies shown in FIG. 4 and FIG. 3 may include ratcheting type systems, where adjustment (e.g., tightening) may be achieved by simply pulling an adjustment end through a ratcheting buckle. As the adjustment end is pulled through the buckle, teeth of tracks may facilitate tightening, while preventing motion in a loosening direction. Release may be facilitated by pulling up on the buckle thereby releasing the engagement with the teeth of tracks causing the belt to loosen.

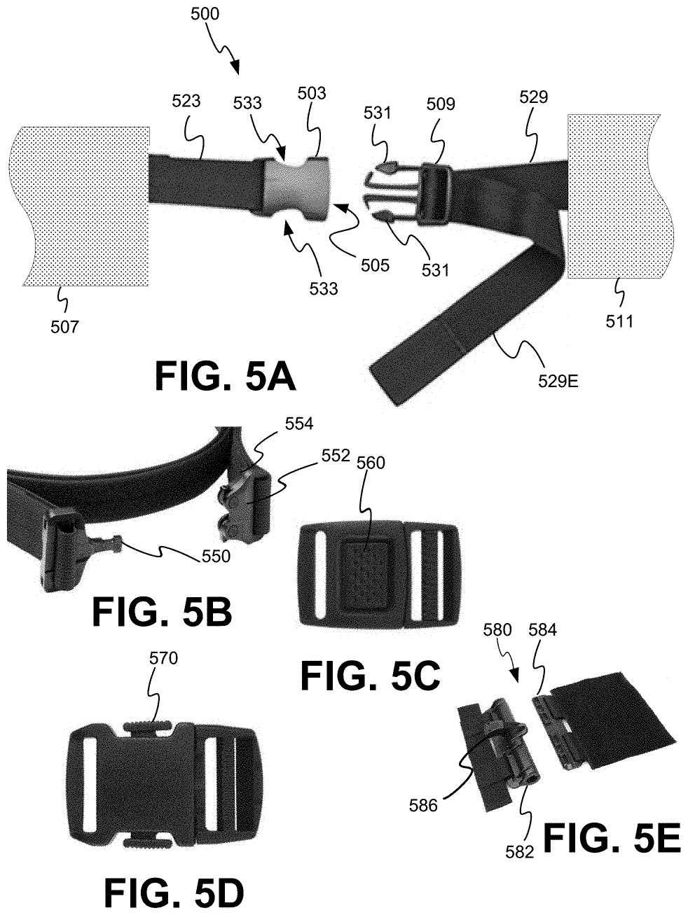

FIG. 5A is a diagram an adjustable fastening assembly 500 that can be used with support structure 200 (FIG. 2), according to still other embodiments. In these embodiments, adjustable fastening assembly 500 includes a side squeeze release mechanism similar to those used in strap applications (e.g., as used in backpacks and other outdoor equipment).

In embodiments, adjustable fastening assembly 500 includes a receptacle 503 and a member or plug 509. In some embodiments, receptacle 503 is fixedly attached to one end of a strap 523. The other end of strap 523 is fixedly attached to a portion 507 of the support structure (corresponding to portion 207 in FIG. 2). Although strap 523 is shown in FIG. 5 being attached to an edge portion 507, in embodiments portion 507 can extend out to and even beyond receptacle 503. Embodiments of receptacle 503 can be made from materials described above for receptacle 303 (FIG. 3).

Member 509 has a buckle or slide through which a strap 529 is threaded to adjustably attach member 509 to strap 529. Strap 529 has one end fixedly attached to a portion 511 of the support structure (similar to portion 211 of support structure 200 in FIG. 2). In some embodiments, member 509 can be made from materials described above for track 313 (FIG. 3). Although strap 529 is shown in FIG. 5A being attached to an edge of portion 511, in embodiments portion 511 can extend out to and even beyond member 509. As shown in FIG. 5A, strap 529 has an end 529E that extends beyond member 509 when threaded through member 509.

To wear the support structure, the patient can fasten portions 507 and 511 together by inserting tines 531 of member 509 into opening 505 of receptacle 503. Tines 531 are sized and arranged to occupy a space with a width slightly larger than the width of opening 505, but with a height and depth that does fit into opening 505. Tines 531 are made of resilient material that enables the patient to insert tines 531 into opening 505. Receptacle 503 has side openings 533 into which latch structures at the end of tines 531 fit when member 509 is fully inserted into receptacle 503. The resiliency of tines 531 enable a portion of the latch structures to extend out through side openings 533 so that when the patient wishes to unfasten portion 507 from portion 511, the patient can easily squeeze tines 531 so that the latch structures are forced back into the cavity of receptacle 503, and then withdraw member 509 from receptacle 503.

To adjust the fit of the support structure to tighten it, the patient can pull end 529E of strap 529 through the buckle of member 509, which effectively shortens the distance between member 509 and where it is attached to portion 511. The folding of strap 529 by approximately 180 degrees in the buckle holds the strap in place. To relax the fit of the support structure, the patient, rotates the buckle of member 509 so that the folding of strap 529 in the buckle is reduced enough so that member 509 can be moved along strap 529. The patient can move member 509 relax or untighten the fit by moving member 509 along strap 529 to increase the distance between member 509 and where it is attached to portion 511. A patient can easily perform such adjustments without having to look at receptacle 503 or member 509, which can be advantageous when the patient is wearing the support structure under his or her clothing.

FIGS. 5B-5E are some additional examples of an adjustable fastening assembly that can be used with support structure 200 (FIG. 2), according to still other embodiments. Some of examples may include a single post 550 configured to be inserted into a latch 552 shown in FIG. 5B. Release may be facilitated by a couple of rotational tabs 554. Examples of these types of fastening assemblies may be available from Blue Alfa LLC of Newman, Ga.

Some more examples may include a center button 560 release type as shown in FIG. 5C. Release may be facilitated by depressing the center button 560. Examples of these types of fastening assemblies may be available from Shin Fang Plastic Industrial Co., LTD. of Taichung City, Taiwan.

Some further examples may include two button 570 release type as shown in FIG. 5D. Release may be facilitated by depressing the two buttons 570 on top and bottom of the fastening assembly. Examples of these types of fastening assemblies may be available from Shin Fang Plastic Industrial Co., LTD. of Taichung City, Taiwan.

In some further examples may include a tubular coupling 580 as shown in FIG. 5E. In FIG. 5E, a tube part 582 may be configured to slidably couple with a shaft part 584, where the shaft part 584 may be configured to slide into the tube part 582. Release may be facilitated by pulling on a pull tab 586 and sliding the shaft part 584 or the tube part 582 apart. Examples of these types of fastening assemblies may be available from Blaze Defense Systems of Pelham, Ala.

FIG. 6 is a schematic diagram of components of an example wearable cardioverter defibrillator (WCD) system, according to embodiments. Such a WCD system can include one or more adjustable fastening assemblies described above and may protect an ambulatory patient by electrically restarting their heart if needed. Such a WCD system may have a number of components. These components can be provided separately as modules that can be interconnected, or can be combined with other components, and so on. Although a WCD embodiment is described below, in other embodiments other types of wearable medical devices are used.

FIG. 6 depicts a patient 682. Patient 682 may also be referred to as a person and/or wearer since the patient is wearing components of the WCD system. Patient 682 is ambulatory, which means that, while wearing the wearable portion of the WCD system, patient 682 can walk around and is not necessarily bed ridden. While patient 682 may be considered to be also a “user” of the WCD system, this is not a requirement. For instance, a user of the wearable cardioverter defibrillator (WCD) may also be a clinician such as a doctor, nurse, emergency medical technician (EMT) or other similarly tasked individual or group of individuals. In some cases, a user may even be a bystander. The particular context of these and other related terms within this description should be interpreted accordingly.

A WCD system according to embodiments can be configured to defibrillate the patient who is wearing the designated parts the WCD system. Defibrillating can be by the WCD system delivering an electrical charge to the patient's body in the form of an electric shock. The electric shock can be delivered in one or more pulses.

In particular, FIG. 6 also depicts components of a WCD system made according to embodiments. One such component is a support structure 670 that is wearable by ambulatory patient 682. Accordingly, support structure 670 is configured to be worn by ambulatory patient 682 for at least several hours per day, and for at least several days, even a few months. It will be understood that support structure 670 is shown only generically in FIG. 6, and in fact partly conceptually. FIG. 6 is provided merely to illustrate concepts about support structure 670 and is not to be construed as limiting how support structure 670 is implemented, or how it is worn. In accordance with embodiments of the present disclosure, support structure 670 also includes one or more adjustable fastening assemblies (not shown in FIG. 6) as described above in conjunction with FIGS. 2-5.

Support structure 670 can be implemented in many different ways. For example, it can be implemented in a single component or a combination of multiple components. In embodiments, support structure 670 could include a vest, a half-vest, a garment, etc. In such embodiments such items can be worn similarly to analogous articles of clothing. In embodiments, support structure 670 could include a harness, one or more belts or straps, etc. In such embodiments, such items can be worn by the patient around the torso, hips, over the shoulder, etc. In embodiments, support structure 670 can include a container or housing, which can even be waterproof. In such embodiments, the support structure can be worn by being attached to the patient's body by adhesive material, for example as shown and described in U.S. Pat. No. 8,024,037. Support structure 670 can even be implemented as described for the support structure of US Pat. App. No. US2017/0056682, which is incorporated herein by reference. Of course, in such embodiments, the person skilled in the art will recognize that additional components of the WCD system can be in the housing of a support structure instead of being attached externally to the support structure, for example as described in the US2017/0056682 document. There can be other examples.

FIG. 6 shows a sample external defibrillator 600. As described in more detail later in this document, some aspects of external defibrillator 600 include a housing and an energy storage module within the housing. As such, in the context of a WCD system, defibrillator 600 is sometimes called a main electronics module. The energy storage module can be configured to store an electrical charge. Other components can cause at least some of the stored electrical charge to be discharged via electrodes through the patient, so as to deliver one or more defibrillation shocks through the patient.

FIG. 6 also shows sample defibrillation electrodes 604, 608, which are coupled to external defibrillator 600 via electrode leads 605. Defibrillation electrodes 604, 608 can be configured to be worn by patient 682 in a number of ways. For instance, defibrillator 600 and defibrillation electrodes 604, 608 can be coupled to support structure 670, directly or indirectly. In other words, support structure 670 can be configured to be worn by ambulatory patient 682 so as to maintain at least one of electrodes 604, 608 on the body of ambulatory patient 682, while patient 682 is moving around, etc. The electrode can be thus maintained on the body by being attached to the skin of patient 682, simply pressed against the skin directly or through garments, etc. In some embodiments the electrode is not necessarily pressed against the skin but becomes biased that way upon sensing a condition that could merit intervention by the WCD system. In addition, many of the components of defibrillator 600 can be considered coupled to support structure 670 directly, or indirectly via at least one of defibrillation electrodes 604, 608.

When defibrillation electrodes 604, 608 make good electrical contact with the body of patient 682, defibrillator 600 can administer, via electrodes 604, 608, a brief, strong electric pulse 611 through the body. Pulse 611 is also known as shock, defibrillation shock, therapy, electrotherapy, therapy shock, etc. Pulse 611 is intended to go through and restart heart 685, in an effort to save the life of patient 682. Pulse 611 can further include one or more pacing pulses of lesser magnitude to simply pace heart 685 if needed, and so on.

A prior art defibrillator typically decides whether to defibrillate or not based on an ECG signal of the patient. However, external defibrillator 600 may initiate defibrillation, or hold-off defibrillation, based on a variety of inputs, with the ECG signal merely being one of these inputs.

A WCD system according to embodiments can obtain data from patient 682. For collecting such data, the WCD system may optionally include at least an outside monitoring device 680. Device 680 is called an “outside” device because it could be provided as a standalone device, for example not within the housing of defibrillator 600. Device 680 can be configured to sense or monitor at least one local parameter. A local parameter can be a parameter of patient 682, or a parameter of the WCD system, or a parameter of the environment, as will be described later in this document.

For some of these parameters, device 680 may include one or more sensors or transducers. Each one of such sensors can be configured to sense a parameter of patient 682, and to render an input responsive to the sensed parameter. In some embodiments the input is quantitative, such as values of a sensed parameter; in other embodiments the input is qualitative, such as informing whether or not a threshold is crossed, and so on. Sometimes these inputs about patient 682 are also referred to herein as patient physiological inputs and patient inputs. In embodiments, a sensor can be construed more broadly, as encompassing many individual sensors.

Optionally, device 680 is physically coupled to support structure 670. In addition, device 680 may be communicatively coupled with other components that are coupled to support structure 670. Such communication can be implemented by a communication module, as will be deemed applicable by a person skilled in the art in view of this description.

In embodiments, one or more of the components of the shown WCD system may be customized for patient 682. This customization may include a number of aspects. For instance, support structure 670 can be fitted to the body of patient 682. For another instance, baseline physiological parameters of patient 682 can be measured, such as the heart rate of patient 682 while resting, while walking, motion detector outputs while walking, etc. The measured values of such baseline physiological parameters can be used to customize the WCD system, in order to make its diagnoses more accurate, since patients' bodies differ from one another. Of course, such parameter values can be stored in a memory of the WCD system, and so on. Moreover, a programming interface can be made according to embodiments, which receives such measured values of baseline physiological parameters. Such a programming interface may input automatically in the WCD system these, along with other data.

FIG. 7 is a diagram showing components of an external defibrillator 700, made according to embodiments. These components can be, for example, included in external defibrillator 600 of FIG. 6. The components shown in FIG. 7 can be provided in a housing 701, which may also be referred to as casing 701.

External defibrillator 700 is intended for a patient who would be wearing it, such as ambulatory patient 682 of FIG. 6. Defibrillator 700 may further include a user interface 780 for a user 782. User 782 can be patient 682, also known as wearer 682. Or, user 782 can be a local rescuer at the scene, such as a bystander who might offer assistance, or a trained person. Or, user 782 might be a remotely located trained caregiver in communication with the WCD system.

User interface 780 can be made in a number of ways. User interface 780 may include output devices, which can be visual, audible, or tactile, for communicating to a user by outputting images, sounds or vibrations. Images, sounds, vibrations, and anything that can be perceived by user 782 can also be called human-perceptible indications (HPIs). There are many examples of output devices. For example, an output device can be a light, or a screen to display what is sensed, detected and/or measured, and provide visual feedback to rescuer 782 for their resuscitation attempts, and so on. Another output device can be a speaker, which can be configured to issue voice prompts, beeps, loud alarm sounds and/or words to warn bystanders, etc.

User interface 780 may further include input devices for receiving inputs from users. Such input devices may include various controls, such as pushbuttons, keyboards, touchscreens, one or more microphones, and so on. An input device can be a cancel switch, which is sometimes called an “I am alive” switch or “live man” switch. In some embodiments, actuating the cancel switch can prevent the impending delivery of a shock.

Defibrillator 700 may include an internal monitoring device 781. Device 781 is called an “internal” device because it is incorporated within housing 701. Monitoring device 781 can sense or monitor patient parameters such as patient physiological parameters, system parameters and/or environmental parameters, all of which can be called patient data. In other words, internal monitoring device 781 can be complementary or an alternative to outside monitoring device 680 of FIG. 6. Allocating which of the parameters are to be monitored by which of monitoring devices 680, 781 can be done according to design considerations. Device 781 may include one or more sensors, as also described elsewhere in this document.

Patient parameters may include patient physiological parameters. Patient physiological parameters may include, for example and without limitation, those physiological parameters that can be of any help in detecting by the WCD system whether or not the patient needs a shock or other intervention or assistance. Patient physiological parameters may also optionally include the patient's medical history, event history and so on. Examples of such parameters include the patient's ECG, blood oxygen level, blood flow, blood pressure, blood perfusion, pulsatile change in light transmission or reflection properties of perfused tissue, heart sounds, heart wall motion, breathing sounds and pulse. Accordingly, monitoring devices 680, 781 may include one or more sensors configured to acquire patient physiological signals. Examples of such sensors or transducers include one or more electrodes to detect ECG data, a perfusion sensor, a pulse oximeter, a device for detecting blood flow (e.g. a Doppler device), a sensor for detecting blood pressure (e.g. a cuff), an optical sensor, illumination detectors and sensors perhaps working together with light sources for detecting color change in tissue, a motion sensor, a device that can detect heart wall movement, a sound sensor, a device with a microphone, an SpO2 sensor, and so on. In view of this disclosure, it will be appreciated that such sensors can help detect the patient's pulse, and can therefore also be called pulse detection sensors, pulse sensors, and pulse rate sensors. In addition, a person skilled in the art may implement other ways of performing pulse detection.

In some embodiments, the local parameter is a trend that can be detected in a monitored physiological parameter of patient 782. A trend can be detected by comparing values of parameters at different times over short and long terms. Parameters whose detected trends can particularly help a cardiac rehabilitation program include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2, CO2, or other parameters such as those mentioned above, f) respiratory function, respiratory rate, etc.; g) motion, level of activity; and so on. Once a trend is detected, it can be stored and/or reported via a communication link, along perhaps with a warning if warranted. From the report, a physician monitoring the progress of patient 782 will know about a condition that is either not improving or deteriorating.

Patient state parameters include recorded aspects of patient 782, such as motion, posture, whether they have spoken recently plus maybe also what they said, and so on, plus optionally the history of these parameters. Or, one of these monitoring devices could include a location sensor such as a Global Positioning System (GPS) location sensor. Such a sensor can detect the location, plus a speed can be detected as a rate of change of location over time. Many motion detectors output a motion signal that is indicative of the motion of the detector, and thus of the patient's body. Patient state parameters can be very helpful in narrowing down the determination of whether SCA is indeed taking place.

A WCD system made according to embodiments may thus include a motion detector. In embodiments, a motion detector can be implemented within monitoring device 680 or monitoring device 781. Such a motion detector can be made in many ways as is known in the art, for example by using an accelerometer. In this example, a motion detector 787 is implemented within monitoring device 781. A motion detector of a WCD system according to embodiments can be configured to detect a motion event. A motion event can be defined as is convenient, for example a change in motion from a baseline motion or rest, etc. In such cases, a sensed patient parameter is motion.

System parameters of a WCD system can include system identification, battery status, system date and time, reports of self-testing, records of data entered, records of episodes and intervention, and so on. In response to the detected motion event, the motion detector may render or generate, from the detected motion event or motion, a motion detection input that can be received by a subsequent device or functionality.

Environmental parameters can include ambient temperature and pressure. Moreover, a humidity sensor may provide information as to whether or not it is likely raining. Presumed patient location could also be considered an environmental parameter. The patient location could be presumed, if monitoring device 680 or 781 includes a GPS location sensor as per the above, and if it is presumed that the patient is wearing the WCD system.

Defibrillator 700 typically includes a defibrillation port 710, which can be a socket in housing 701. Defibrillation port 710 includes electrical nodes 714, 718. Leads of defibrillation electrodes 704, 708, such as leads 605 of FIG. 6, can be plugged into defibrillation port 710, so as to make electrical contact with nodes 714, 718, respectively. It is also possible that defibrillation electrodes 704, 708 are connected continuously to defibrillation port 710, instead. Either way, defibrillation port 710 can be used for guiding, via electrodes, to the wearer at least some of the electrical charge that has been stored in an energy storage module 750 that is described more fully later in this document. The electric charge will be the shock for defibrillation, pacing, and so on.

Defibrillator 700 may optionally also have a sensor port 719 in housing 701, which is also sometimes known as an ECG port. Sensor port 719 can be adapted for plugging in sensing electrodes 709, which are also known as ECG electrodes and ECG leads. It is also possible that sensing electrodes 709 can be connected continuously to sensor port 719, instead. Sensing electrodes 709 are types of transducers that can help sense an ECG signal, e.g., a 12-lead signal, or a signal from a different number of leads, especially if they make good electrical contact with the body of the patient and in particular with the skin of the patient. As with defibrillation electrodes 704, 708, the support structure can be configured to be worn by patient 782 so as to maintain sensing electrodes 709 on a body of patient 782. For example, sensing electrodes 709 can be attached to the inside of support structure 670 for making good electrical contact with the patient, similarly with defibrillation electrodes 704, 708.

Optionally a WCD system according to embodiments also includes a fluid that it can deploy automatically between the electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid may be in the form of a low-viscosity gel, so that it does not flow away, after being deployed, from the location it is released near the electrode. The fluid can be used for both defibrillation electrodes 704, 708, and for sensing electrodes 709.

The fluid may be initially stored in a fluid reservoir, not shown in FIG. 7. Such a fluid reservoir can be coupled to the support structure. In addition, a WCD system according to embodiments further includes a fluid deploying mechanism 774. Fluid deploying mechanism 774 can be configured to cause at least some of the fluid to be released from the reservoir and be deployed near one or both of the patient locations to which electrodes 704, 708 are configured to be attached to the patient. In some embodiments, fluid deploying mechanism 774 is activated prior to the electrical discharge responsive to receiving activation signal AS from a processor 730, which is described more fully later in this document.

In some embodiments, defibrillator 700 also includes a measurement circuit 720, as one or more of its working together with its sensors or transducers. Measurement circuit 720 senses one or more electrical physiological signals of the patient from sensor port 719, if provided. Even if defibrillator 700 lacks sensor port 719, measurement circuit 720 may optionally obtain physiological signals through nodes 714, 718 instead, when defibrillation electrodes 704, 708 are attached to the patient. In these cases, the input reflects an ECG measurement. The patient parameter can be an ECG, which can be sensed as a voltage difference between electrodes 704, 708. In addition, the patient parameter can be an impedance, which can be sensed between electrodes 704, 708 and/or between the connections of sensor port 719 considered pairwise. Sensing the impedance can be useful for detecting, among other things, whether these electrodes 704, 708 and/or sensing electrodes 709 are not making good electrical contact with the patient's body. These patient physiological signals may be sensed when available. Measurement circuit 720 can then render or generate information about them as inputs, data, other signals, etc. As such, measurement circuit 720 can be configured to render a patient input responsive to a patient parameter sensed by a sensor. In some embodiments, measurement circuit 720 can be configured to render a patient input, such as values of an ECG signal, responsive to the ECG signal sensed by sensing electrodes 709. More strictly speaking, the information rendered by measurement circuit 720 is output from it, but this information can be called an input because it is received as an input by a subsequent device or functionality.

Defibrillator 700 also includes a processor 730. Processor 730 may be implemented in a number of ways in various embodiments. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 730 may include, or have access to, a non-transitory storage medium, such as memory 738 that is described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

Processor 730 can be considered to have a number of modules. One such module can be a detection module 732. Detection module 732 can include a Ventricular Fibrillation (VF) detector. The patient's sensed ECG from measurement circuit 720, which can be available as inputs, data that reflect values, or values of other signals, may be used by the VF detector to determine whether the patient is experiencing VF. Detecting VF is useful because VF typically results in SCA. Detection module 732 can also include a Ventricular Tachycardia (VT) detector, and so on.

Another such module in processor 730 can be an advice module 734, which generates advice for what to do. The advice can be based on outputs of detection module 732. There can be many types of advice according to embodiments. In some embodiments, the advice is a shock/no shock determination that processor 730 can make, for example via advice module 734. The shock/no shock determination can be made by executing a stored Shock Advisory Algorithm. A Shock Advisory Algorithm can make a shock/no shock determination from one or more ECG signals that are captured according to embodiments and determine whether or not a shock criterion is met. The determination can be made from a rhythm analysis of the captured ECG signal or otherwise.

In some embodiments, when the determination is to shock, an electrical charge is delivered to the patient. Delivering the electrical charge is also known as discharging and shocking the patient. As mentioned above, such can be for defibrillation, pacing, and so on.

In ideal conditions, a very reliable shock/no shock determination can be made from a segment of the sensed ECG signal of the patient. In practice, however, the ECG signal is often corrupted by electrical noise, which makes it difficult to analyze. Too much noise sometimes causes an incorrect detection of a heart arrhythmia, resulting in a false alarm to the patient. Noisy ECG signals may be handled as described in U.S. patent application Ser. No. 16/037,990, filed on Jul. 17, 2018 and since published as US 2019/0030351 A1, and also in U.S. patent application Ser. No. 16/038,007, filed on Jul. 17, 2018 and since published as US 2019/0030352 A1, both by the same applicant and incorporated herein by reference.

Processor 730 can include additional modules, such as other module 736, for other functions. In addition, if internal monitoring device 781 is indeed provided, processor 730 may receive its inputs, etc.

Defibrillator 700 optionally further includes a memory 738, which can work together with processor 730. Memory 738 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. Memory 738 is thus a non-transitory storage medium. Memory 738, if provided, can include programs for processor 730, which processor 730 may be able to read and execute. More particularly, the programs can include sets of instructions in the form of code, which processor 730 may be able to execute upon reading. The programs may also include other information such as configuration data, profiles, scheduling etc. that can be acted on by the instructions. Executing is performed by physical manipulations of physical quantities, and may result in functions, operations, processes, acts, actions and/or methods to be performed, and/or the processor to cause other devices or components or blocks to perform such functions, operations, processes, acts, actions and/or methods. The programs can be operational for the inherent needs of processor 730 and can also include protocols and ways that decisions can be made by advice module 734. In addition, memory 738 can store prompts for user 782 if this user is a local rescuer. Moreover, memory 738 can store data. This data can include patient data, system data and environmental data, for example as learned by internal monitoring device 781 and outside monitoring device 680. The data can be stored in memory 738 before it is transmitted out of defibrillator 700 or be stored there after it is received by defibrillator 700.

Defibrillator 700 can optionally include a communication module 790, for establishing one or more wired or wireless communication links with other devices of other entities, such as a remote assistance center, Emergency Medical Services (EMS), and so on. The communication links can be used to transfer data and commands. The data may be patient data, event information, therapy attempted, CPR performance, system data, environmental data, and so on. For example, communication module 790 may transmit wirelessly, e.g., on a daily basis, heart rate, respiratory rate, and other vital signs data to a server accessible over the internet, for instance as described in U.S. Published Patent App. Pub. No. 20140043149A1 entitled “MOBILE COMMUNICATION DEVICE & APP FOR WEARABLE DEFIBRILLATOR SYSTEM”. This data can be analyzed directly by the patient's physician and can also be analyzed automatically by algorithms designed to detect a developing illness and then notify medical personnel via text, email, phone, etc. Module 790 may also include such interconnected sub-components as may be deemed necessary by a person skilled in the art, for example an antenna, portions of a processor, supporting electronics, outlet for a telephone or a network cable, etc.

Defibrillator 700 may also include a power source 740. To enable portability of defibrillator 700, power source 740 typically includes a battery. Such a battery is typically implemented as a battery pack, which can be rechargeable or not. Sometimes a combination is used of rechargeable and non-rechargeable battery packs. Other embodiments of power source 740 can include an AC power override, for where AC power will be available, an energy-storing capacitor, and so on. Appropriate components may be included to provide for charging or replacing power source 740. In some embodiments, power source 740 is controlled and/or monitored by processor 730.

Defibrillator 700 may additionally include an energy storage module 750. Energy storage module 750 can be coupled to the support structure of the WCD system, for example either directly or via the electrodes and their leads. Module 750 is where some electrical energy can be stored temporarily in the form of an electrical charge, when preparing it for discharge to administer a shock. In embodiments, module 750 can be charged from power source 740 to the desired amount of energy, as controlled by processor 730. In typical implementations, module 750 includes a capacitor 752, which can be a single capacitor or a system of capacitors, and so on. In some embodiments, energy storage module 750 includes a device that exhibits high power density, such as an ultracapacitor. As described above, capacitor 752 can store the energy in the form of an electrical charge, for delivering to the patient.

A decision to shock can be made responsive to the shock criterion being met, as per the above-mentioned determination. When the decision is to shock, processor 730 can be configured to cause at least some or all of the electrical charge stored in module 750 to be discharged through patient 682 while the support structure is worn by patient 682, so as to deliver a shock 611 to patient 682.

For causing the discharge, defibrillator 700 moreover includes a discharge circuit 755. When the decision is to shock, processor 730 can be configured to control discharge circuit 755 to discharge through the patient at least some of all of the electrical charge stored in energy storage module 750. Discharging can be to nodes 714, 718, and from there to defibrillation electrodes 704, 708, so as to cause a shock to be delivered to the patient. Circuit 755 can include one or more switches 757. Switches 757 can be made in a number of ways, such as by an H-bridge, and so on. Circuit 755 could also be thus controlled via processor 730, and/or user interface 780.

A time waveform of the discharge may be controlled by thus controlling discharge circuit 755. The amount of energy of the discharge can be controlled by how much energy storage module has been charged, and also by how long discharge circuit 755 is controlled to remain open.

Defibrillator 700 can optionally include other components.

A person skilled in the art will be able to practice the present invention after careful review of this description, which is to be taken as a whole. Details have been included to provide a thorough understanding. In other instances, well-known aspects have not been described, in order to not obscure unnecessarily this description.

Some technologies or techniques described in this document may be known. Even then, however, it is not known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, component, or process that are identical or at least similar or related. Where made, such a further effort was not required, but was nevertheless made gratuitously so as to accelerate comprehension by the reader. Even where made in this document, such a further effort might not have been made completely consistently for all of the versions or embodiments that are made possible by this description. Accordingly, the description controls in defining an item, aspect, component, or process, rather than its reference numeral. Any similarity in reference numerals may be used to infer a similarity in the text, but not to confuse aspects where the text or other context indicates otherwise.

The claims of this document define certain combinations and subcombinations of elements, features and acts or operations, which are regarded as novel and non-obvious. The claims also include elements, features and acts or operations that are equivalent to what is explicitly mentioned. Additional claims for other such combinations and subcombinations may be presented in this or a related document. These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that the claim can have one or more of this component or this item.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

What is claimed is:

1. A wearable cardioverter defibrillator (WCD) system, comprising:
a support structure configured to be worn by a patient using the WCD system;
an energy storage device configured to store electric charge for providing therapy to the patient;
an output circuit coupled to the energy storage device;
one or more sensors coupled to the support structure;
one or more therapy electrodes coupled to the support structure and the output circuit;
a processor configured to:
receive one or more output signals from the one or more sensors,
determine at least in part based on the received one or more output signals whether the patient has a shockable arrhythmia,
responsive to a determination that the patient has a shockable arrhythmia, cause electric charge from the energy storage device to be delivered to the patient via the output circuit and the therapy electrodes; and
an adjustable ratchet-type fastener, attached to the support structure, configured to removably fasten first and second portions of the support structure to each other and further configured to be adjusted by the patient to adjust a fit of the support structure on the patient while the patient is wearing the wearable medical device.

2. The WCD system of claim 1, wherein the ratchet-type fastener has an adjustment increment between ⅛ inch to ½ inch.

3. The WCD system of claim 1, wherein the ratchet-type fastener is further configured to enable a patient wearing the wearable medical device to adjust the fit of the support structure without unfastening the first and second portions from each other.

4. The WCD system of claim 1, wherein the ratchet-type fastener is further configured to enable a patient wearing the wearable medical device to adjust the fit of the support structure while wearing clothing over the support structure and without having to adjust the clothing to view or access the ratchet-type fastener.

5. The WCD system of claim 1, wherein the ratchet-type fastener comprises a side-squeeze mechanism configured to disengage a pawl from a tooth of the ratchet-type fastener.

6. A wearable medical device, comprising:
a support structure;
one or more sensors coupled to the support structure;
circuitry operatively coupled to the one or more sensors to receive one or more output signals from the one or more sensors; and
an adjustable ratchet-type fastener, attached to the support structure, configured to removably fasten first and second portions of the support structure to each other and further configured to be adjustable by a patient to adjust a fit of the support structure on the patient while the patient is wearing the wearable medical device.

7. The wearable medical device of claim 6, wherein the ratchet-type fastener has an adjustment increment between ⅛ inch to ½ inch.

8. The wearable medical device of claim 6, wherein the ratchet-type fastener is further configured to enable a patient wearing the wearable medical device to adjust the fit of the support structure without unfastening the first and second portions from each other.

9. The wearable medical device of claim 6, wherein the ratchet-type fastener is further configured to enable a patient wearing the wearable medical device to adjust the fit of the support structure while wearing clothing over the support structure and without having to adjust the clothing to view or access the ratchet-type fastener.

10. The wearable medical device of claim 6, wherein the one or more sensors comprise ECG sensors.

11. The wearable medical device of claim 6, wherein the ratchet-type fastener comprises a side-squeeze mechanism configured to disengage a pawl from a tooth of the ratchet-type fastener.

12. A wearable medical device, comprising:
a support structure;
one or more sensors coupled to the support structure;
circuitry operatively coupled to the one or more sensors to receive one or more output signals from the one or more sensors; and
an adjustable cinch-type fastener, attached to the support structure, configured to removably fasten first and second portions of the support structure to each other and further configured be adjustable by a patient to adjust a fit of the support structure on the patient while the patient is wearing the wearable medical device.

13. The wearable medical device of claim 12, wherein the cinch-type fastener is substantially continuously adjustable.

14. The wearable medical device of claim 12, wherein the cinch-type fastener is further configured to enable a patient wearing the wearable medical device to adjust the fit of the support structure without unfastening the first and second portions from each other.

15. The wearable medical device of claim 12, wherein the cinch-type fastener is further configured to enable a patient wearing the wearable medical device to adjust the fit of the support structure while wearing clothing over the support structure and without having to adjust the clothing to view or access the cinch-type fastener.

16. The wearable medical device of claim 12, wherein the wearable medical device comprises a wearable cardioverter defibrillator.

17. The wearable medical device of claim 12, wherein the one or more sensors comprise ECG sensors.
